(19) United States
(12) Patent Application Publication
(10) Pub. No.: US 2025/0232443 A1
(43) Pub. Date: Jul. 17, 2025

(54) MULTI-INPUT AND/OR MULTI-OUTPUT VIRTUAL STAINING

(71) Applicant: Carl Zeiss AG, Oberkochen (DE)

(72) Inventors: Alexander FREYTAG, Erfurt (DE); Christian KUNGEL, Penzberg (DE)

(73) Assignee: Carl Zeiss AG, Oberkochen (DE)

(21) Appl. No.: 18/850,747

(22) PCT Filed: Mar. 30, 2021
(86) PCT No.: PCT/EP2021/058270

§ 371 Date: Sep. 25, 2024

(30) Foreign Application Priority Data
Mar. 30, 2020 (DE) 10 2020 108 745.4

(51) Int. Cl. G06T 7/00; G06T 5/50; G06V 20/69; G16H 30/20

(52) U.S. Cl. CPC G06T 7/0014; G06T 5/50; G06V 20/695; G16H 30/20; G06T 2207/10056; G06T 2207/10064; G06T 2207/20081; G06T 2207/20221; G06T 2207/30024

(57) ABSTRACT

A method of virtual staining of a tissue sample includes obtaining multiple sets of imaging data. The imaging data depicts a tissue sample and has been acquired using multiple imaging modalities. Further, the method includes fusing and processing the multiple sets of imaging data in a machine-learning logic. The machine-learning logic is configured to provide at least one output image. Each one of the at least one output image depicts the tissue sample comprising a respective virtual stain.

### FIELD OF THE INVENTION

Various embodiments relate to techniques for virtual staining by utilizing a machine-learning logic. Various examples specifically relate to processing multiple sets of imaging data acquired using multiple imaging modalities. Further, various examples relate to outputting multiple output images depicting a tissue sample including multiple virtual stains.

### BACKGROUND OF THE INVENTION

Histopathology is an important tool in the diagnosis of a disease. Histopathology refers to the optical examination of tissue samples. Diagnosis of cells in the tissue sample is facilitated.

Typically, histopathological examination starts with surgery, biopsy, or autopsy for obtaining the tissue to be examined. The tissue may be processed to remove water and to prevent decay. The processed sample may then be embedded in a wax block. From the wax block, thin sections may be cut. Said thin sections may be referred to as tissue samples hereinafter.

The tissue samples may be analyzed by a histopathologist in a microscope. The tissue samples may be stained with a chemical stain using an appropriate staining laboratory process, to thereby facilitate the analysis of the tissue sample. In particular, chemical stains may reveal cellular components which are very difficult to observe in the unstained tissue sample. Moreover, chemical stains may provide contrast. The chemical stains may highlight one or more biomarkers or predefined structures of the tissue sample.

The most commonly used chemical stain in histopathology is a combination of haematoxylin and eosin (abbreviated H&E). Haematoxylin is used to stain nuclei blue, while eosin stains cytoplasm and the extracellular connective tissue matrix pink. There are hundreds of various other techniques which have been used to selectively stain cells. Recently, antibodies have been used to stain particular proteins, lipids and carbohydrates. Called immunohistochemistry, this technique has greatly increased the ability to specifically identify categories of cells under a microscope. Staining with an H&E stain may be considered as common gold standard for histopathologic diagnosis.

By coloring tissue samples with chemical stains, otherwise almost transparent and indistinguishable structures/tissue sections of the tissue samples become visible for the human eye. This allows pathologists and researchers to investigate the tissue sample under a microscope or with a digital bright-field equivalent image and assess the tissue morphology (structure) or to look for the presence or prevalence of specific cell types, structures or even microorganisms such as bacteria.

Preferably, several chemical stains are used to fully assess the pathology case. Typically, only one chemical stain can be applied to a tissue sample. Thus, if several chemical stains are required for diagnosis, several tissue samples have to be prepared. Moreover, different chemical stains may require different staining protocols. Thus, the known chemical staining techniques are labour- and cost-intensive.

WO 2019/154987 A1 discloses a method providing a virtually stained image looking like a typical image of a tissue sample which has been stained with a conventional chemical stain using a machine-learning logic. Virtual-staining techniques bypasses the typically labor-intensive and costly histological staining procedures, and could be used as a blueprint for the virtual staining of tissue images acquired with other label-free imaging modalities. Virtual-staining approaches could be used for microguiding molecular analysis at the unstained-tissue level, by locally identifying regions of interest on the basis of virtual staining, and by using this information to guide subsequent analysis of the tissue, for example, microimmunohistochemistry or sequencing. This type of virtual microguidance on an unlabeled tissue sample might facilitate the high-throughput identification of disease subtypes and the development of customized therapies for patients.

### SUMMARY OF THE INVENTION

There is a need for advanced techniques of virtual staining. In particular, there is a need for techniques which allow accurate virtual staining and/or flexible virtual staining.

According to one aspect of the invention, a method of virtual staining of a tissue sample includes obtaining multiple sets of imaging data. The multiple sets of imaging data depict a tissue sample and have been acquired using multiple imaging modalities. Further, the method includes fusing and processing the multiple sets of imaging data in a machine-learning logic. The machine-learning logic is configured to provide at least one output image. Each one of the at least one output image depicts the tissue sample including a respective virtual stain.

Tissue samples may relate to thin sections of the wax block comprising an embedded processed sample as described hereinbefore. However, the term tissue sample may also refer to tissue having been processed differently or not having been processed at all. For example, tissue sample may refer to a part of tissue observed in vivo and/or tissue excised from a human, an animal or a plant, wherein the observed tissue sample has been further processed ex vivo, e.g., prepared using a frozen section method. A tissue sample may be any kind of a biological sample. The term tissue sample may also refer to a cell, which cell can be of procaryotic or eucaryotic origin, a plurality of procaryotic and/or eucaryotic cells such as an array of single cells, a plurality of adjacent cells such as a cell colony or a cell culture, a complex sample such as a biofilm or a microbiome that contains a mixture of different procaryotic and/or eucaryotic cell species and/or an organoid.

According to another aspect of the invention, a computer-program product or a computer program or a computer-readable storage medium or a data signal includes program code. The program code can be loaded and executed by at least one circuit. Upon executing the program code, the at least one circuit performs a method of virtual staining of a tissue sample includes obtaining multiple sets of imaging data. The imaging data depicts a tissue sample and has been acquired using multiple imaging modalities. Further, the method includes fusing and processing the multiple sets of imaging data in a machine-learning logic. The machine-learning logic is configured to provide at least one output image. Each one of the at least one output image depicts the tissue sample including a respective virtual stain.

According to yet another aspect of the invention, a device includes a circuit. The circuit is configured to obtain multiple sets of imaging data. The multiple sets of imaging data depict a tissue sample and have been acquired using multiple imaging modalities. Further, the circuit is configured to fuse and process the multiple sets of imaging data in a machine-learning logic. The machine-learning logic is configured to provide at least one output image. Each one of the at least one output image depicts the tissue sample including a respective virtual stain.

According to yet another aspect of the invention, a method is used to perform a training of a machine-learning logic for virtual staining. The machine-learning logic includes at least one encoder branch and multiple decoder branches. The method includes obtaining one or more training images. The one or more training images depict one or more tissue samples. Further, the method includes obtaining multiple reference images. The multiple reference images depict the one or more tissue samples including multiple chemical stains. Also, the method includes processing the one or more training images in the machine-learning logic. The machine-learning logic provides multiple training output images for each one of the one or more training images. Each one of the multiple training output images is associated with a respective decoder branch and depicts the respective tissue sample including a respective virtual stain. Further, the method includes performing the training of the machine-learning logic by updating parameter values of the machine-learning logic based on a comparison between such reference images and training output images that are associated with corresponding chemical stains and virtual stains.

The term chemical staining may also comprise modifying molecules of any one of the different types of tissue sample mentioned above. The modification may lead to fluorescence under a certain illumination (e.g., an illumination under ultra-violet (UV) light). For example, chemical staining may include modifying genetic material of the tissue sample. Chemically stained tissue samples may comprise transfected cells. Transfection may refer to a process of deliberately introducing naked or purified nucleic acids into eukaryotic cells. It may also refer to other methods and cell types. It may also refer to non-viral DNA transfer in bacteria and non-animal eukaryotic cells, including plant cells.

Modifying genetic material of the tissue sample may make the genetic material observable using a certain image modality. For example, the genetic material may be rendered fluorescent. In some examples, modifying genetic material of the tissue sample may cause the tissue sample to produce molecules being observable using a certain image modality. For example, modifying genetic material of the tissue sample may induce the production of fluorescent proteins by the tissue sample.

According to another aspect of the invention, a computer-program product or a computer program or a computer-readable storage medium or a data signal includes program code. The program code can be loaded and executed by at least one circuit. Upon executing the program code, the at least one circuit performs a method of performing a training of a machine-learning logic for virtual staining. The machine-learning logic includes at least one encoder branch and multiple decoder branches. The method includes obtaining one or more training images. The one or more training images depict one or more tissue samples. Further, the method includes obtaining multiple reference images. The multiple reference images depict the one or more tissue samples comprising multiple chemical stains. Also, the method includes processing the one or more training images in the machine-learning logic. The machine-learning logic provides multiple training output images for each one of the one or more training images. Each one of the multiple training output images is associated with a respective decoder branch and depicts the respective tissue sample including a respective virtual stain. Besides, the method includes performing the training of the machine-learning logic by updating parameter values of the machine-learning logic based on a comparison between such reference images and training output images that are associated with corresponding chemical stains and virtual stains.

According to yet another aspect of the invention, a device comprises a circuit. The circuit is configured to perform a training of a machine-learning logic for virtual staining. The machine-learning logic comprises at least one encoder branch and multiple decoder branches. The circuit is configured to obtain one or more training images. The one or more training images depict one or more tissue samples. Further, the circuit is configured to obtain multiple reference images. The multiple reference images depict the one or more tissue samples comprising multiple chemical stains. Also, the circuit is configured to process the one or more training images in the machine-learning logic. The machine-learning logic provides multiple training output images for each one of the one or more training images. Each one of the multiple training output images is associated with a respective decoder branch and depicts the respective tissue sample comprising a respective virtual stain. Besides, the circuit is configured to perform the training of the machine-learning logic by updating parameter values of the machine-learning logic based on a comparison between such reference images and training output images that are associated with corresponding chemical stains and virtual stains.

According to yet another aspect of the invention, a method of virtual staining of a tissue sample includes obtaining at least one set of imaging data. The imaging data depicts a tissue sample. Further, the method includes fusing and processing the at least one set of imaging data in a machine-learning logic. The machine-learning logic is configured to provide multiple output images. Each one of the multiple output image depicts the tissue sample including a respective virtual stain. A respective computer program or computer-program product or computer readable storage medium or a data signal including program code that is executable by a circuit to perform this method is provided. A respective device is provided that includes a circuit to execute this method.

It is to be understood that the features mentioned above and those yet to be explained below may be used not only in the respective combinations indicated, but also in other combinations or in isolation without departing from the scope of the invention.

### BRIEF DESCRIPTION OF THE DRAWINGS

FIG. 1 schematically illustrates a workflow for staining a tissue sample according to various examples.

FIG. 2 schematically illustrates images depicting a tissue sample with and without a chemical or virtual stain according to various examples.

FIG. 3 is a flowchart of a method according to various examples, the method enabling inference of one or more output images depicting a tissue sample including one or more virtual stains.

FIG. 4 schematically illustrates a tissue sample and multiple sets of imaging data depicting the tissue sample according to various examples.

FIG. 5 schematically illustrates a machine-learning logic according to various examples.

FIG. 6 schematically illustrates an example implementation of the machine-learning logic according to various examples.

FIG. 7 schematically illustrates an example implementation of the machine-learning logic according to various examples.

FIG. 8 schematically illustrates an example implementation of the machine-learning logic according to various examples.

FIG. 9 is a flowchart of a method according to various examples, the method enabling training of a machine-learning logic for virtual staining according to various examples.

FIG. 10 schematically illustrates aspects with respect to the training of the machine-learning logic according to various examples.

### DETAILED DESCRIPTION OF THE INVENTION

Some examples of the present disclosure generally provide for a plurality of circuits or other electrical devices. All references to the circuits and other electrical devices and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, machine-learning-specific hardware, e.g., a graphics processor unit (GPU) and/or a tensor processing unit (TPU), integrated circuits, memory devices (e.g. FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a set of program code that is embodied in a non-transitory computer readable medium programmed to perform any number of the functions as disclosed.

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are taken to be illustrative only.

The drawings are to be regarded as being schematic representations and elements illustrated in the drawings, which are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various techniques described herein generally relate to machine learning. Machine learning, especially deep learning, provides a data-driven strategy to solve problems. Classic inference techniques are able to extract patterns from data based on hand-designed features, to solve problems; an example technique would be regression. However, such classic inference techniques heavily depend on the accurate choice for the hand-designed features, which choice depends on the designer's ability. One solution to such a problem is to utilize machine learning to discover not only the mapping from features to output, but also the features themselves. This is as training of a machine-learning logic.

Various techniques described herein generally relate to virtual staining of a tissue sample by utilizing a trained machine-learning logic (MLL). The MLL can be implemented, e.g., by a support vector machine or a deep neural network which includes at least one encoder branch and at least one decoder branch.

More specifically, according to various examples, multiple sets of imaging data can be fused and processed by the MLL. This is referred to as a multi-input scenario.

Alternatively or additionally to such a multi-input scenario, multiple virtually stained images can be obtained (labeled output images hereinafter), from the trained MLL; the multiple virtually stained images can depict the tissue sample including different virtual stains. This is referred to as a multi-output scenario.

As a general rule, examples as summarized in TAB. 1 below can be implemented.

TABLE 1
Various scenarios for input and output of the MLL

| | Brief | |
|---|---|---|
| Scenario | description | Details |
| A | Single-input | The MLL can include multiple decoder |
| | multi-output | branches to provide the multiple outputs, |
| | (SIMO) | but include a single encoder branch to |
| | | receive respective input imaging data. By |
| | | providing multiple output images, it is |
| | | possible to depict the tissue sample |
| | | including multiple virtual stains, thereby |
| | | facilitating an accurate diagnosis. |
| B | Multi-input | The MLL can include multiple encoder |
| | multi-output | branches, or even fuse multiple sets of |
| | (MIMO) | imaging data at the input layer. For the |
| | | decoder part of the MLL, see scenario A. By |
| | | considering multiple sets of imaging data, |
| | | it is possible to more accurately determine |
| | | the multiple output images. |
| C | Multi-input | The MLL can include multiple encoder |
| | single-output | branches, or even fuse multiple sets of |
| | (MISO) | imaging data at the input layer. By |
| | | considering multiple sets of imaging data, |
| | | it is possible to more accurately determine |
| | | the single output image. |

The one or more output images depict the tissue sample including respective virtual stains, i.e., the output images can have a similar appearance as respective images depicting the tissue sample including a corresponding chemical stain. Thus, the virtual stain can have a correspondence in a chemical stain of a tissue sample stained using a staining laboratory process.

For example, the MLL can generate virtual H&E (Hematoxylin and Eosin) stained images of the tissue sample, and/or virtually stained images of the tissue sample highlighting HER2 (human epidermal growth factor receptor 2) proteins and/or ERBB2 (Erb-B2 Receptor Tyrosine Kinase 2) genes.

Another example would pertain to virtual fluorescence staining. For example, in life-science applications, images of cells—e.g., arranged ex-vivo in a multi-well plate—are acquired using transmitted-light microscopy. Also, a reflected light microscope may be used, e.g., in an endoscope or as a surgical microscope. It is then possible to selectively stain certain cell organelles, e.g., nucleus, ribosomes, the endoplasmic reticulum, the golgi apparatus, chloroplasts, or the mitochondria. A fluorophore (or fluorochrome, similarly to a chromophore) is a fluorescent chemical compound that can re-emit light upon light excitation. Fluorophores can be used to provide a fluorescence chemical stain. By using different fluorophores, different chemical stains can be achieved. For example, a Hoechst stain would be a fluorescent dye that can be used to stain DNA. Other fluorophores include 5-aminolevulinic acid (5-ALA), fluorescein, and Indocyanine green (ICG) that can even be used in-vivo. Fluorescence can be selectively excited by using light in respective wavelengths; the fluorophores then emit light at another wavelength. Respective fluorescence microscopes use respective light sources. It has been observed that illumination using light to excite fluorescence can harm the sample; this is avoided when providing virtual fluorescence staining. The virtual fluorescence staining mimics the fluorescence chemical staining, without exposing the tissue to respective excitation light.

By using a multi-input scenario (cf. TAB. 1, scenario B and C), an increased accuracy for processing the imaging data in the MLL can be achieved. This is because by using multiple sets of imaging data that have been acquired using multiple imaging modalities, different biomarkers or biological structures can be highlighted in each one of the multiple sets.

By using multi-output scenario (cf. TAB. 1, scenario A and B), a tailored virtual stain or a tailored set of multiple virtual stains can be provided such that a pathologist is enabled to provide an accurate analysis. For example, multiple output images depicting the tissue samples having multiple virtual stains may be helpful to provide a particular accurate diagnosis, e.g., based on multiple types of structures and multiple biomarkers being highlighted in the multiple output images, or multiple organelles of the cells being highlighted.

As a general rule, multi-input scenarios may or may not be combined with multi-output scenarios; and likewise, multi-output scenarios may or may not be combined with multi-input scenarios.

As a general rule, imaging data of the tissue sample, as used herein, refers to any kind of data, in particular digital imaging data, representing the tissue sample or parts thereof. For example, depending on the image modality, the dimensionality of the imaging data of the tissue sample may vary. The imaging data may be two-dimensional (2-D), one-dimensional (1-D) or even three-dimensional (3-D). Different sets of imaging data can have different dimensionality and/or resolution. If more than one image modality is used for obtaining imaging data, a first set of the imaging data may be two-dimensional and another set of the imaging data may be one-dimensional or three-dimensional. For instance, microscopy imaging may provide imaging data that includes images having spatial resolution, i.e., including multiple pixels. Scanning through the tissue sample with a confocal microscope may provide imaging data comprising three-dimensional voxels. Spectroscopy of the tissue sample may result in imaging data providing spectral information of the whole tissue sample without spatial resolution. In another embodiment, spectroscopy of the tissue sample may result in imaging data providing spectral information for several positions of the tissue sample which results in imaging data comprising spatial resolution but being sparsely sampled.

As a general rule, imaging modalities, as used herein, may include, e.g., imaging of the tissue sample in one or more specific spectral bands, in particular, spectral bands in the ultra violet, visible and/or infrared range (multi-spectral microscopy). Imaging modalities may also comprise a Raman analysis of the tissue samples, in particular a stimulated Raman scattering (SRS) analysis of the tissue sample, a coherent anti-Stokes Raman scattering, CARS, analysis of the tissue sample, a surface enhanced Raman scattering, SERS, analysis of the tissue sample. Further, the imaging modalities may include a fluorescence analysis of the tissue sample, in particular, fluorescence lifetime imaging microscopy, FLIM, analysis of the tissue sample. The imaging modality may prescribe a phase sensitive acquisition of the digital imaging data. The imaging modality may also prescribe a polarization sensitive acquisition of the digital imaging data. Digital phase contrast is a further example of imaging modality. Yet a further example would be transmitted-light or reflected-light microscopy, e.g., for observing cells. Imaging modalities may, as a general rule, imaging tissue in-vivo or ex-vivo. An endoscope may be used to acquire images in-vivo, e.g., a confocal microscope or using endoscopic optical coherence tomography (e.g., scanned or full-field). A confocal fluorescence scanner could be used. Endoscopic two-photon microscopy would be a further imaging modality. A surgical microscope may be used; the surgical microscope may, itself provide for multiple imaging modalities, e.g., microscopic images or fluorescence images, e.g., in specific spectral bands or combinations of two or more wavelengths, or even hyperspectral images.

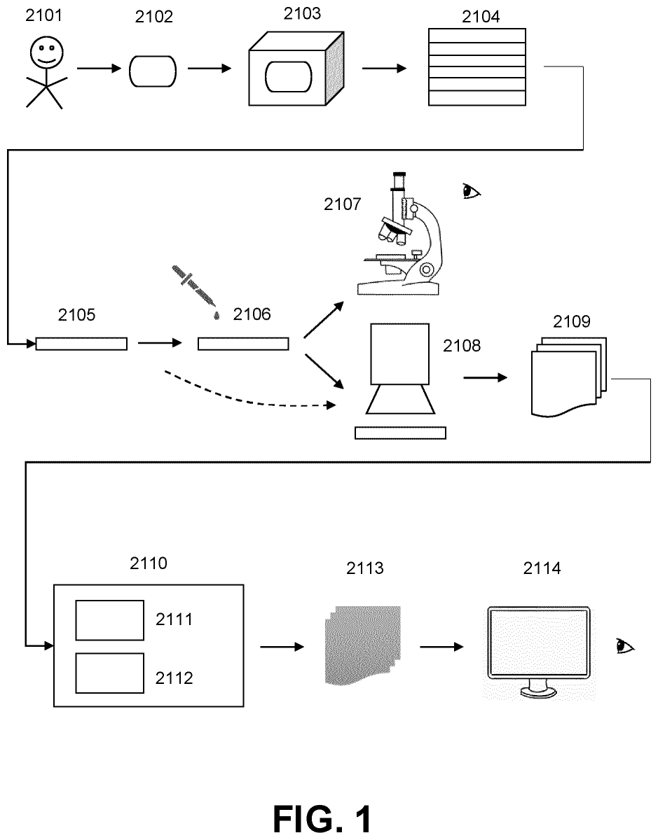

FIG. 1 illustrates aspects with respect to a workflow for generating images depicting a tissue sample including a stain, e.g., a chemical stain or a virtual stain. FIG. 1 schematically illustrates an example of a histopathology workflow. As explained above, virtual staining can also be applied in other use cases than histopathology. Then, different workflows for generating images can be applicable. For instance, for fluorescence imaging of cells, tissue samples including cell samples may be otherwise acquired and imaged in a respective microscope. Also, in-vivo imaging using an endoscope would be a possible use case for generating imaging data of tissue samples.

As shown in FIG. 1, for histopathology, tissue 2102 may be obtained from a living creature 2101 by surgery, biopsy or autopsy. After some processing steps to remove water and to prevent decay, said tissue 2102 may be embedded in a wax block 2103. From said block 2103, a plurality of slices 2104 may be obtained for further analysis. One slice of said plurality of slices 2104 may also be called a tissue sample 2105. Corresponding tissue samples 2015 may be obtained from adjacent slices.

As mentioned before, the tissue could also include cell samples or in-vivo inspection using, e.g., a surgical microscope or an endoscope.

Before analyzing the tissue sample 2105, a chemical stain may optionally be applied to the tissue sample 2105 using a staining laboratory process, to obtain a chemically-stained tissue sample 2106. In some examples, the tissue sample 2105 may also be directly analyzed (dashed arrow in FIG. 1). A chemically stained tissue sample 2106 may facilitate the analysis. In particular, chemical stains may reveal cellular components or generally well-defined structures of cells, which are difficult to observe in the unstained tissue sample 2105. Moreover, chemical stains may provide an increased contrast.

Applying a chemical stain may include a-priori transfecting or direct application of a fluorophore such as 5-ALA.

Traditionally, the tissue sample 2105 or 2106 is analyzed by an expert using a bright field microscope 2107.

Meanwhile, it has become more common to use image acquisition systems 2108 configured for acquiring digital image data of the tissue sample 2105 or the chemically stained tissue sample 2106 using one or more imaging modalities. Using different imaging modalities may facilitate acquiring imaging data 2109—e.g., 1-D, 2-D, or 3-D imaging data—of the tissue sample 2105. The imaging data may, e.g., include images acquired using multispectral microscopy, i.e., having a contrast sensitive to light in one or more specific spectral bands, in particular, spectral bands in the ultra violet, visible and/or infrared range. Imaging modalities may also comprise a Raman analysis of the tissue samples, in particular a stimulated Raman scattering (SRS) analysis of the tissue sample, a coherent anti-Stokes Raman scattering, CARS, analysis of the tissue sample, a surface enhanced Raman scattering, SERS, analysis of the tissue sample. Further, the imaging modalities may comprise a fluorescence analysis of the tissue sample, in particular, fluorescence lifetime imaging microscopy (FLIM), analysis of the tissue sample. The imaging modality may prescribe a phase sensitive acquisition of the digital imaging data. The imaging modality may also prescribe a polarization sensitive acquisition of the digital imaging data.

The imaging data 2109 may be processed in a tissue analyzer 2110. The tissue analyzer 2110 may be implemented by a computer and/or by cloud processing at a server. The tissue analyzer 2110 may include a memory circuitry 2111 for storing the digital image data 2109 and/or program code, and may include a circuit 2112 for processing the digital image data 2109—e.g., upon loading the program code. The tissue analyzer 2110 may process the imaging data 2109 to provide one or more output images 2113 which may be displayed on a display 2114 to be analyzed by an examiner. For example, multiple output images 2113 depicting the tissue sample 2105, 2106 including different virtual stains may be provided (cf. TAB. 1, scenarios A and B). The tissue analyzer 2110 may comprise different types of trained or untrained machine-learning logic (details are with respect to the machine-learning logic are described below) for analyzing the non-stained tissue sample 2105 and/or the chemically stained tissue sample 2106 (i.e., the processor 2112 can execute the machine-learning logic). The output images 2113 may depict the tissue sample 2105 with one or more virtual stains. The image acquisition system 2108 may be used for providing training data and/or reference images as a ground truth for training said machine-learning logic.

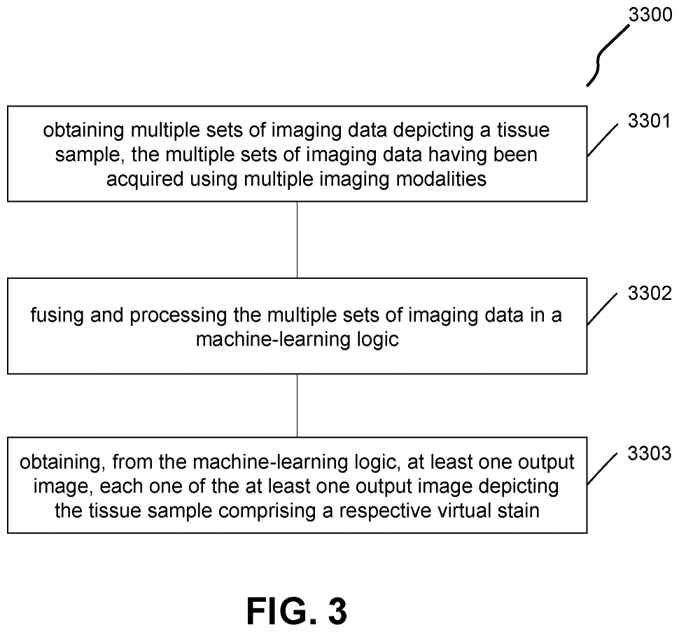

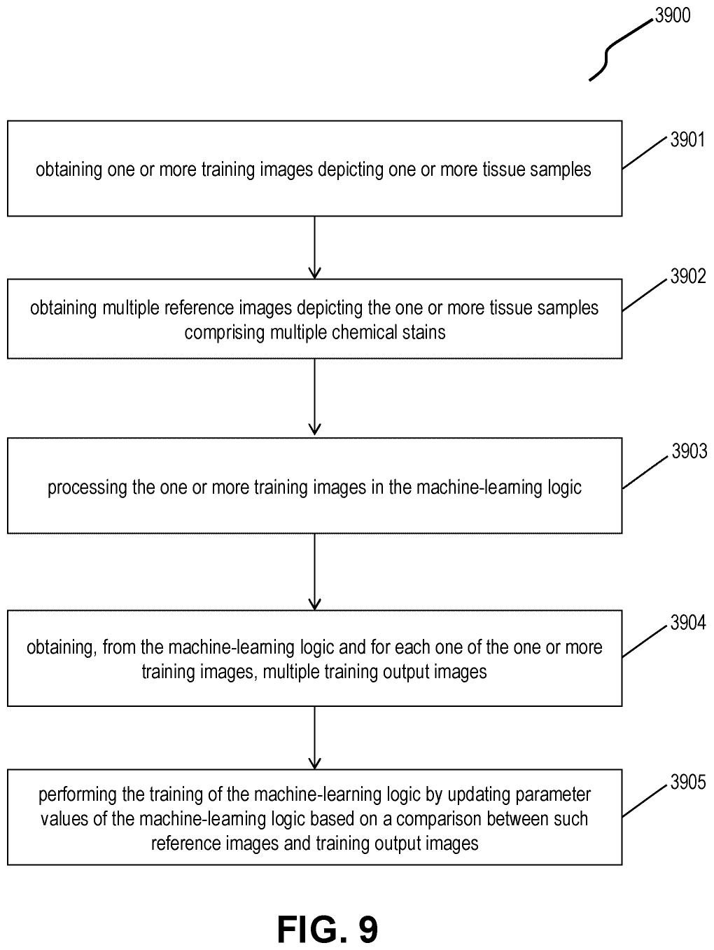

More generally, the tissue analyzer 2110 includes the circuit 2112 which may include a CPU and/or a GPU and/or a TPU. The circuit 2112 can load program code from the memory 2111. The circuit 2112 can execute the program code. Upon executing the program code, the circuit 2112 can perform one or more of the following logic operations as described throughout this disclosure: obtaining imaging data, e.g., via an input/output (I/O) interface of the tissue analyzer 2204 or by loading the imaging data from the memory; virtual staining of the tissue sample depicted by the imaging data; executing a machine-learning logic (e.g., the machine-learning logic 3500 described in this invention) to process the imaging data (inference); obtain at least one output image, from the machine-learning logic/when executing the machine-learning logic, e.g., to output the at least one output image via the I/O interface; setting parameters or hyper-parameters of the machine-learning logic 3500 when training the machine-learning logic; training the machine-learning logic, etc., For example, the method of FIG. 3 and/or the method of FIG. 9 could be executed by the circuit upon loading the program code.

While in the scenario of FIG. 1 a scenario is shown in which the imaging data 2109 depicts the tissue sample 2106 including the chemical stain, as a general rule, it would be possible that the imaging data 2109 depicts the tissue sample 2106 not including a chemical stain (i.e., the staining is optional, dashed arrow in FIG. 1).

While in the scenario of FIG. 1 only a single imaging modality is used to provide the imaging data 2109, as a general rule, it would be possible to use multiple imaging modalities to provide multiple sets of the imaging data 2109 (cf. TAB. 1: scenarios B and C).

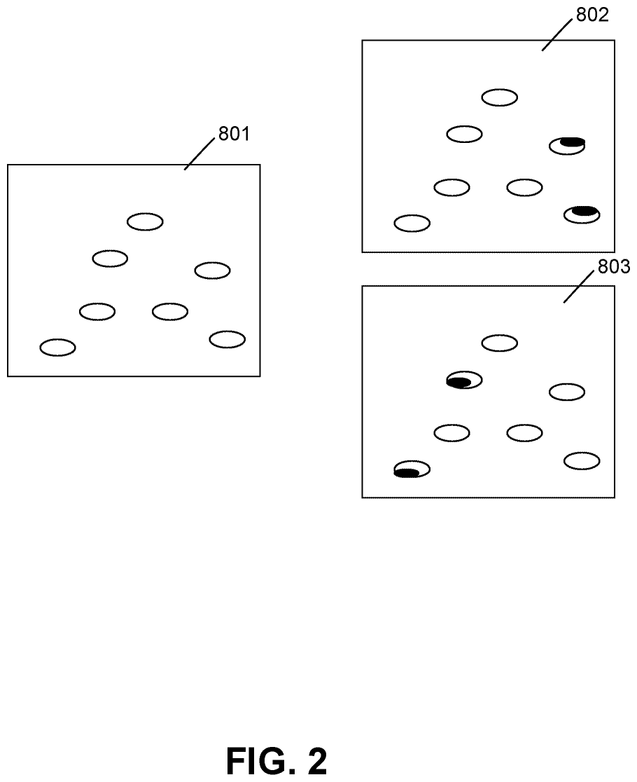

FIG. 2 schematically illustrates images 801-803 depicting a tissue sample. The image 801 depicts the tissue sample not including any chemical or virtual stain. Differently, the image 802 depicts the tissue sample including a chemical or virtual stain. Also, the image 803 depicts the tissue sample including a chemical virtual stain, wherein the chemical virtual stain of the tissue sample depicted by the image 803 is different from the chemical or virtual stain of the tissue sample depicted by the image 802: different structures or biomarker(s) are highlighted (full black areas in FIG. 2).

FIG. 3 is a flowchart of a method 3300 according to various examples. For example, the method 3300 according to FIG. 3 may be executed by at least one circuit—e.g., a CPU and/or a GPU and/or a TPU-upon loading program code from a nonvolatile memory. The method of FIG. 3 may be executed by the tissue analyzer 2110. The method of FIG. 3 facilitates virtual staining of a tissue sample.

FIG. 3 illustrates aspects with respect to virtual staining of a tissue sample. FIG. 3 illustrates aspects with respect to obtaining multiple sets of imaging data depicting the tissue sample by using multiple imaging modalities. FIG. 3 generally relates to multi-input scenario, as described above (cf. TAB. 1, Scenarios B and C). FIG. 3 also illustrates aspects with respect to fusing and processing the multiple sets of imaging data in an MLL, and then obtaining (outputting), from the MLL, at least one output image of which each one depicts the tissue sample including a respective virtual stain.

In detail, at block 3301, multiple sets of imaging data depicting a tissue sample are obtained (e.g., loaded from a memory or obtained via an input interface from a data acquisition unit) and the multiple sets of imaging data are acquired using multiple imaging modalities.

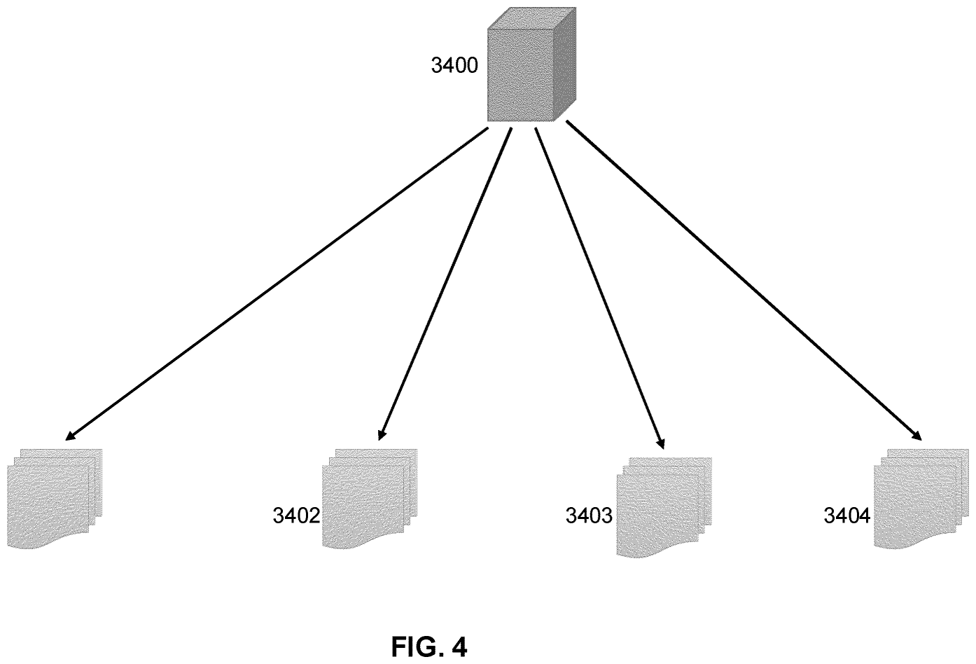

This is explained in connection with FIG. 4. FIG. 4 depicts a tissue sample 3400 and four sets of imaging data of the tissue sample 3400 acquired using four different imaging modalities, i.e., imaging data set 3401, 3402, 3403 and 3404, respectively. Alternatively, the imaging data set 3401, 3402, 3403 and 3404 can be respectively acquired using the same imaging modality but with different imaging settings or parameters, such as different (low or high) magnification levels, etc. Combinations of such scenarios are possible.

Referring again to FIG. 3: Each imaging data set 3401-3404 can include multiple instances of imaging data, e.g., multiple images (cf. FIG. 2: image 801) taken at different positions of the sample (e.g., for stitching) and/or at different times.

The tissue sample 3400 can be a cancer tissue sample removed from a patient, a tissue sample of other animals or plants.

The multiple imaging modalities can be selected from the group including: hyperspectral microscopy imaging, fluorescence imaging, auto-fluorescence imaging, lightsheet imaging, digital phase contrast; Raman spectroscopy; fluorescence lifetime imaging; phase-sensitive imaging; polarization-sensitive imaging; surface-enhanced Raman scattering; stimulated Raman scattering; coherent anti-Stokes Raman scattering, etc. Further imaging modalities have been discussed above.

Depending on the particular imaging modality, a spatial dimensionality of the imaging data of each set 3401-3404 may vary, e.g., 1-D or 2-D or even 3-D. For instance, microscopy imaging or fluorescence imaging may provide imaging data that include images having spatial resolution, i.e., including multiple pixels. Lightsheet imaging may provide 3-D voxels. On the other hand, where Raman spectroscopy is used, it would be possible that an integral signal not possessing spatial resolution is obtained as the respective set of imaging data (corresponding to a 1-D data point); however, also scanning Raman spectroscopy is known where some 2-D spatial resolution can be provided. For instance, digital phase contrast can be generated using multiple illumination directions and digital post-processing to combine images associated with each one of the multiple illumination directions. See, e.g., US 2017 0 276 923 A1.

As a general rule, different imaging modalities may, in some examples, rely on a similar physical observables, e.g., both may pertain to fluorescence imaging or microscopy, but using different acquisition parameters. Example acquisition parameters could include, e.g., illumination type (e.g., brightfield versus dark field microscopy), magnification level, resolution, refresh rate, etc.

Hyperspectral scans help to acquire the substructure of an individual cell to identify subtle changes (morphological change of membrane, change in size of cell components, . . . ). Adjacent z-slices of corresponding tissue samples can be captured in hyperspectral scans, e.g., by scanning through the probe with a confocal microscope (e.g., a light-sheet microscope, LSM), focusing the light-sheet in LSMs to slightly different z-levels. It is possible to acquire adjacent cell information like what happens in widefield microscopy (integral acquisition). A further class of imaging modalities includes molecularly sensitive methods like Raman, coherent Raman (SRS, CARS), SERS, Fluorescence imaging, FLIM, IR-Imaging. This helps to acquire chemical/molecular information. Yet another technique is dynamic cell imaging to acquire cell metabolism information. Yet a further imaging modality includes phase or polarization sensitive imaging to acquire structural information through contrast changes. For instance, digital phase contrast can be generated using multiple illumination directions and digital post-processing to combine images associated with each one of the multiple illumination directions. See, e.g., US 2017 0 276 923 A1.

The method 3300 of FIG. 3 optionally includes pre-processing after obtaining the multiple sets of imaging data, such as one or a combination of the following processing techniques: noise filtering; registration between imaging data of different sets of imaging data, for example, any pairs of the sets, etc.; resizing of the imaging data, etc.

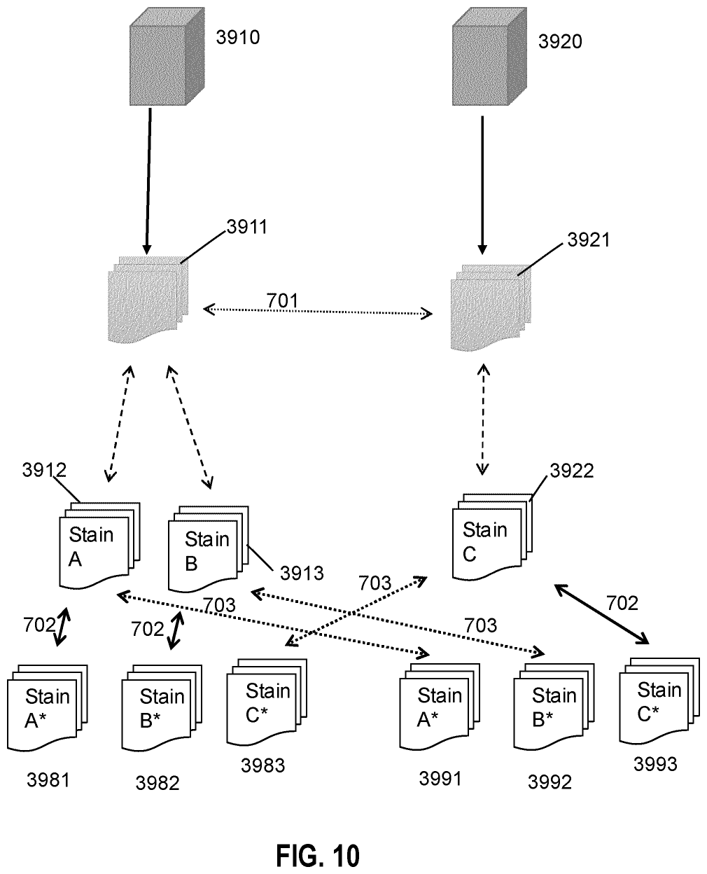

At block 3302, the multiple sets of imaging data are fused and processed by an MLL. The MLL has been trained using supervised learning, semi-supervised learning, or unsupervised learning. A detailed description of an example method of performing training of the MLL will be explained later in connection with FIG. 9 and FIG. 10.

As a general rule, various implementations of the MLL are conceivable. In one example, a deep neural network may be used. For example, a U-net implementation is possible. See Ronneberger, Olaf, Philipp Fischer, and Thomas Brox. “U-net: Convolutional networks for biomedical image segmentation.” International Conference on Medical image computing and computer-assisted intervention. Springer, Cham, 2015.

More generally, the deep neural network can include multiple hidden layers. The deep neural network can include an input layer and an output layer. The hidden layers are arranged in between the input layer and the output layer. There can be a spatial contraction and a spatial expansion implemented by one or more encoder branches and one or more decoder branches, respectively. I.e., the x-y-resolution of respective representations of the imaging data and the output images may be decreased (increased) from layer to layer along the one or more encoder branches (decoder branches). At the same time, feature channels can increase and decrease along the one or more encoder branches and the one or more decoder branches, respectively. The one or more encoder branches and the one or more decoder branches are connected via a bottleneck. At the output layer or layers, the deep neural network can include decoder heads that include an activation function, e.g., a linear or non-linear activation function.

Thus, the MLL can include at least one encoder branch and at least one decoder branch. The at least one encoder branch provides a spatial contraction of respective representatives of the multiple sets of imaging data, and the at least one decoder branch provides a spatial expansion of the respective representatives of the at least one output image.

It is, however, not required in all scenarios that the MLL implements spatial concentration and expansion. In other examples, the spatial resolution may not be affected (possibly with the exception of edge cropping).

As a general rule, the fusing of the multiple sets of imaging data is implemented by concatenation or stacking of the respective representatives of the multiple sets of imaging data at at least one layer of the neural network. This may be an input layer (a scenario sometimes referred to as early fusion or input fusion) or a hidden layer (a scenario sometimes referred to as middle fusion or late fusion). For middle fusion, it would even be possible that the fusing is implemented at the bottleneck (sometimes referred to as bottleneck fusion). Where there are multiple encoder branches, the connection joining the multiple encoder branches defines the layer at which the fusing is implemented. As a general rule, it is possible that fusing of different pairs of imaging data is implemented at different positions, e.g., different layers.

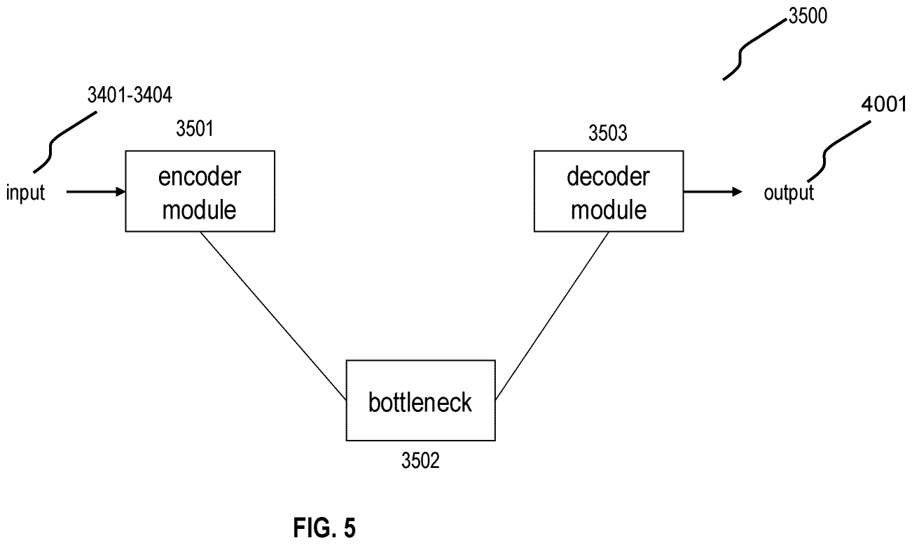

Details with respect to an implementation of the MLL are illustrated in FIG. 5.

FIG. 5 schematically illustrates the MLL 3500 implemented as a deep neural network according to various examples. As an exemplary architecture of the MLL, FIG. 5 schematically illustrates an overview of the MLL 3500. The MLL 3500 includes an encoder module 3501 having at least one encoder branch, a decoder module 3503 having at least one decoder branch, and a bottleneck 3502 for coupling the encoder module 3501 and the decoder module 3503. Each encoder branch of the encoder module 3501, each decoder branch of the decoder module 3503, and the bottleneck 3502 may respectively include at least one block having at least one layer selected from the group including: convolutional layers, activation function layers (e.g., ReLU (rectified linear unit), Sigmoid, tanh, Maxout, ELU (Exponential Linear Unit), scaled Exponential Linear Unit (SELU), Softmax and so on), downsampling layers, upsampling layers, normalization layers (e.g., batch normalization, instance normalization, group normalization, channel normalization, etc.), dropout layers, etc. Thus, each layer defines a respective mathematical operation. Mathematical operations of different layers may be parallelized within each block.

For example, each encoder or decoder branch can include several blocks, each block usually having one or more layers, which may be named as an encoder block and a decoder block, respectively. Every block can include a single layer or multiple layers. It would be possible that within a block, calculations—e.g., for multiple layers—are parallelized.

Encoder branches can be built from encoder blocks followed by downsampler blocks. Downsampler blocks may be implemented by using max-pooling, average-pooling, or strided convolution. Upsampler blocks may be implemented by using transposed-convolution, nearest neighbor interpolation, or bilinear interpolation. We also found it helpful to follow such operations by convolution with activations.

Decoder branches can be built from upsampler blocks followed by decoder blocks. For upsampler blocks, it is possible to apply transposed-convolution, nearest neighbor interpolation, or bilinear interpolation. Especially for the latter two, it has been found that placing several convolution layers thereafter is highly valuable.

More generally, an example encoder or decoder block includes convolutional layers with activation layers and followed by normalization layers. Alternatively, each encoder or decoder block may include more complex blocks, e.g., inception blocks (see, e.g., Szegedy, Christian, et al. “Inception-v4, inception-resnet and the impact of residual connections on learning.” Thirty-first AAAI conference on artificial intelligence. 2017), DenseBlocks (see, e.g., Jégou, S.; Drozdzal, M.; Vazquez, D.; Romero, A. & Bengio, Y., “The One Hundred Layers Tiramisu: Fully Convolutional DenseNets for Semantic Segmentation”, in Proceedings of the IEEE conference on computer vision and pattern recognition workshops (ICCV-WS) 2017), RefineBlocks (see, e.g., Lin, G.; Milan, A.; Shen, C. & Reid, I., “RefineNet: Multi-Path Refinement Networks with Identity Mappings for High-Resolution Semantic Segmentation”, Conference on Computer Vision and Pattern Recognition (CVPR), 2016, arXiv preprint arXiv:1611.09326), or having multiple operations in parallel (e.g., convolution and strided convolution) or having multiple operations after each other (e.g., three convolution with activation and then followed by normalization before going to downsampling), etc.

The encoder module 3501 is fed with the multiple sets of imaging data—e.g., the sets 3401, 3402, 3403 and 3404, cf. FIG. 4—as input. The decoder module 3503 outputs desired one or more output images 4001 depicting the tissue sample including a respective virtual stain (the output images can be labelled virtually stained images; cf. FIG. 2, images 802-803).

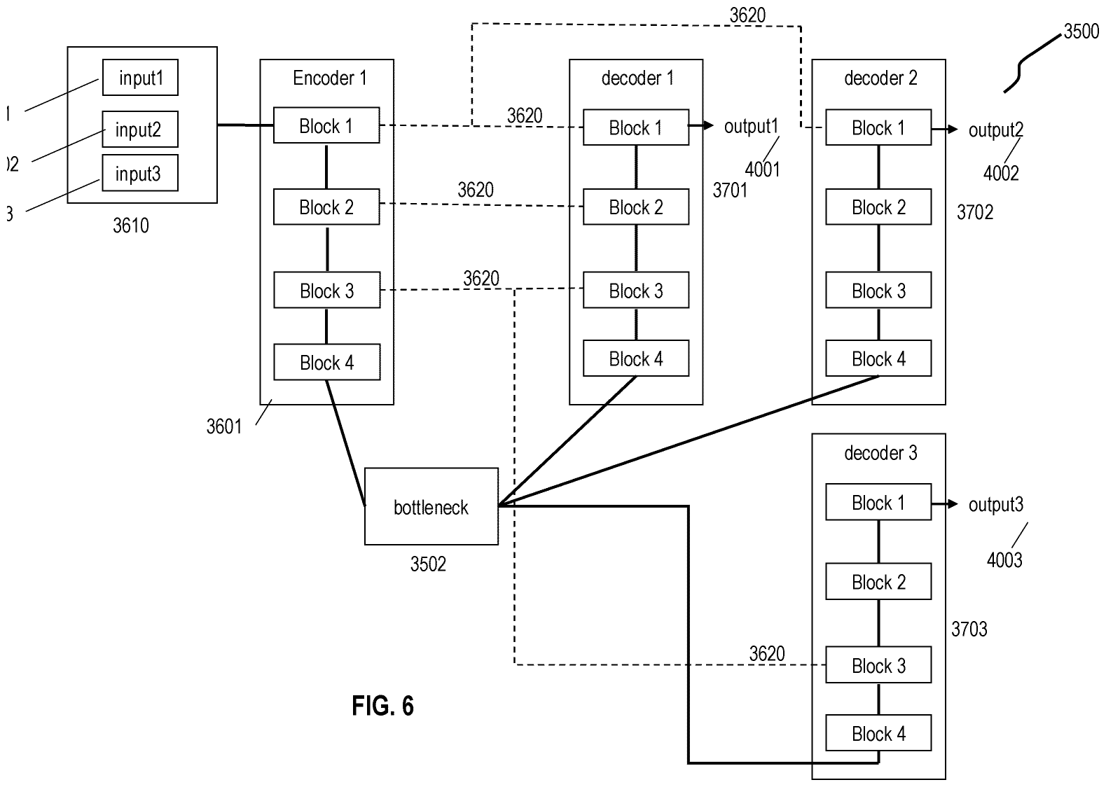

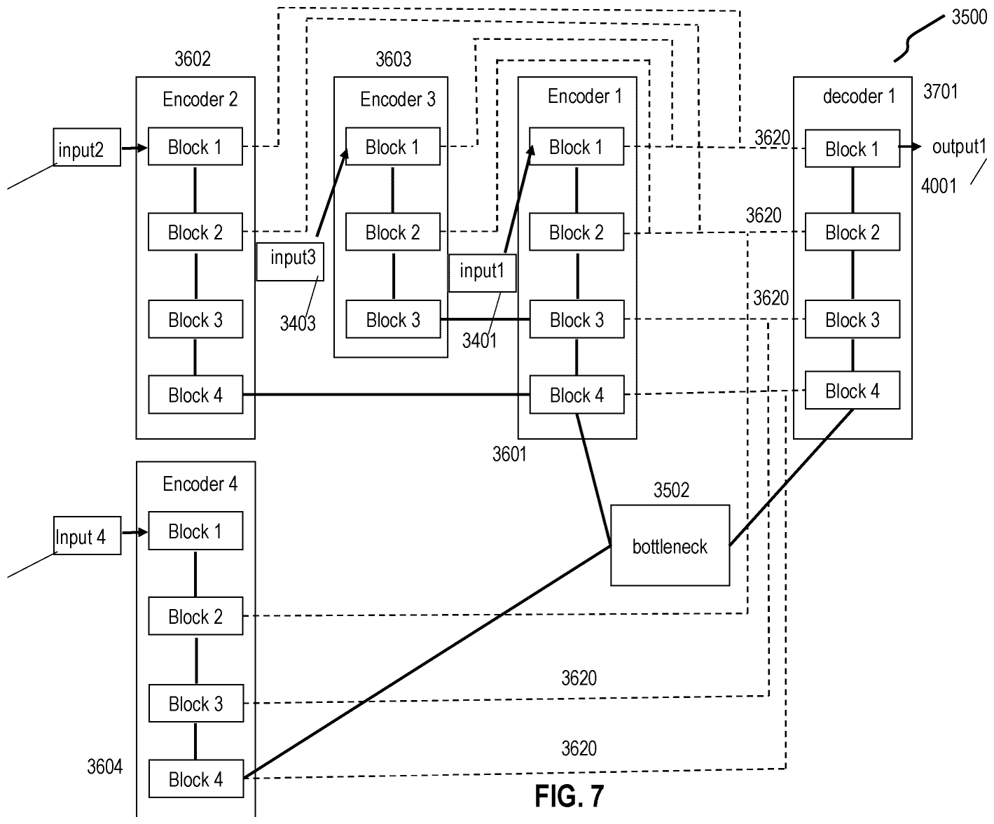

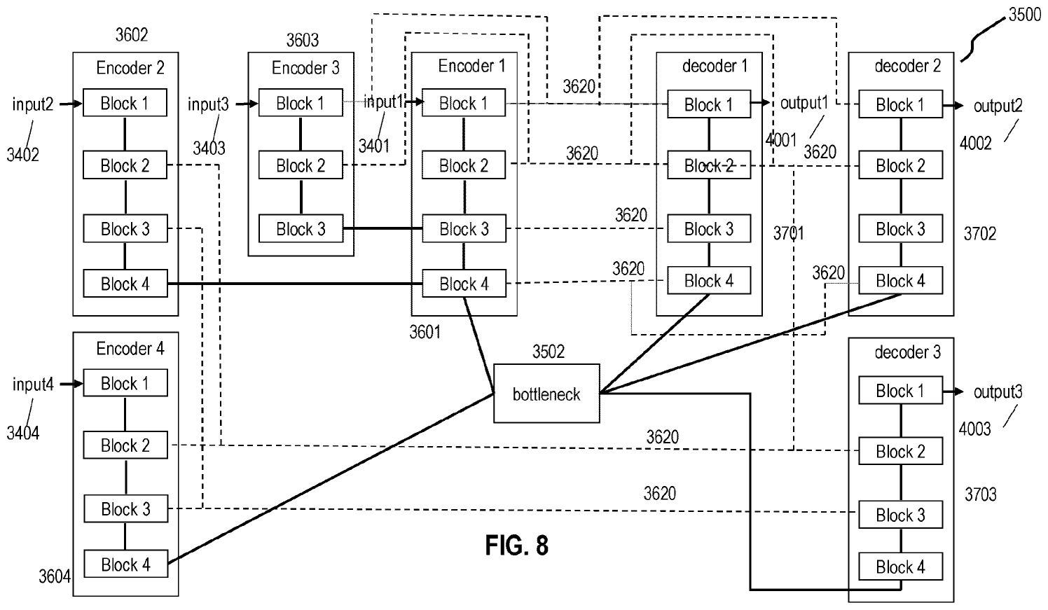

FIG. 6, FIG. 7, and FIG. 8 schematically illustrate details of three exemplary architectures of the MLL 3500. Different architectures of the MLL 3500 can be used to implement different strategies for fusing the multiple sets of imaging data in a multi-input scenario. Further, different architectures of the MLL 3500 can be used to implement multi-output scenarios. The principles of the architectures illustrated in FIG. 6, FIG. 7, and FIG. 8 can be combined with each other: for example, it would be possible that different strategies for fusing the multiple sets of imaging data as illustrated in FIG. 6, FIG. 7, and FIG. 8 are combined with each other, e.g., combining fusing at the input layer with fusing at a hidden layer or at the bottleneck for different sets of imaging data.

With reference to FIG. 6, in the illustrated example, the MLL 3500 includes a single encoder branch 3601, and the fusing of the multiple sets of imaging data (e.g., input1, input2 and input3 may be any three of the four imaging data sets 3401, 3402, 3403 of FIG. 4) is implemented by concatenation at an input layer 3610 of the single encoder branch 3600. The single encoder branch 3600, via the bottleneck 3502, connects to three decoder branches 3701, 3702 and 3703, respectively (albeit it would be possible to have a single decoder branch, for a single-output scenario, cf. TAB. 1, Scenario C). FIG. 6 is, accordingly, a multi-input multi-output scenario using a single encoder branch.

As shown in FIG. 7 and FIG. 8, the MLL 3500 includes multiple encoder branches 3601-3604 and each one of the multiple encoder branches 3601-3604 is fed with a respective one of the multiple sets of imaging data 3401-3404. FIG. 7 and FIG. 8 thus correspond to a multi-input scenario. FIG. 7 is a single-output scenario (cf. TAB. 1, Scenario C), and FIG. 8 is a multi-output scenario (cf. TAB. 1, Scenario B) including multiple decoder branches 3701-3703.

In the illustrated examples of FIG. 7 and FIG. 8, the fusing of the multiple sets of imaging data is implemented by concatenation at at least one hidden layer of the MLL. For example, the extracted features representing the imaging data set 3403 (input3 fed into encoder branch 3603) is fused with the extracted features representing the imaging data set 3401 (input1 fed into encoder branch 3601) at a hidden layer of block 3 of the encoder branch 3601 and then the combination of such two sets of extracted features is fed into block 4 of the encoder branch 3601 for further processing to extract fused features of the combination of the two sets of features. Similarly, the extracted features representing the imaging data set 3402 (input2 fed into encoder branch 3602) is fused with the fused features of the imaging data sets 3401 and 3403 at a hidden layer of block 4 of the encoder branch 3601 and thereby the fused features of the three imaging data sets 3401, 3402 and 3403 are obtained after further processing implemented by block 4 of the encoder 3601.

The MLL includes a bottleneck 3502 in-between the multiple encoder branches 3601-3604 and the at least one decoder branch 3701-3703. In some examples, the fusing of the multiple sets of imaging data 3401-3404 is at least partially implemented by concatenation at the bottleneck 350. This is illustrated in FIG. 7 and FIG. 8, where the extracted features representing the imaging data set 3404 (input4 fed into encoder branch 3604) is fused with the fused features of the three imaging data sets 3401, 3402 and 3403 at the bottleneck 3502 to obtain fused features of the four imaging data sets 3401-3404; then the fused features of the four imaging data sets 3401-3404 are fed into respective encoder branches 3701-3703 to obtain desired virtually stained images 4001-4003.

According to various examples of the invention, the technique of “skip connections” disclosed by Ronneberger etc. (Ronneberger, Olaf, Philipp Fischer, and Thomas Brox. “U-net: Convolutional networks for biomedical image segmentation.” International Conference on Medical image computing and computer-assisted intervention. Springer, Cham, 2015.) is adopted to the MLL 3500. The skip connections provide respective feature sets of the multiple sets of imaging data to corresponding decoders of the MLL 3500 to facilitate feature decoding and pixel-wise information reconstruction on different scales. The bottleneck 3502 and optionally one or more hidden layers are bypassed.

As shown in FIG. 6, several blocks of the encoder branch 3601 directly couple with corresponding blocks of the decoder branches 3701-3703 via skip connections 3620. For example, block 1 of the encoder branch 3601 directly provides its output that represents fused features of the combination of input1, input2 and input3 to block 1 of the decoder branches 3701 and 3702, respectively; block 2 of the encoder branch 3601 directly provides its output which represents second fused features of the combination of input1, input2 and input3 to block 2 of the decoder branch 3701; and block 3 of the encoder branch 3601 provides its output which represents third fused features of the combination of input1, input2 and input3 to block 3 of the decoder branches 3701 and 3703, respectively.

Skip connections 3620 can, in particular, be used where there are multiple encoder branches 3601-3604: with reference to FIG. 7 and FIG. 8, the MLL 3500 includes multiple encoder branches 3601-3604. The MLL 3500 includes skip connections 3620 to feed outputs of hidden layers of at least two of the multiple encoder branches 3601-3604 to inputs of corresponding hidden layers of the at least one decoder branch 3701-3703. For example, as shown in FIG. 7, the MLL 3500 includes one decoder branch 3701 and each block of the decoder branch 3701 is fed with outputs of corresponding blocks of at least two encoder branches among 3601-3604 via skip connections 3620. Similarly, with reference to FIG. 8, the MLL 3500 includes multiple decoder branches 3701-3703 and some blocks of the three decoder branches receive outputs of corresponding blocks of at least two encoder branches among 3601-3604 via skip connections 3620.

Skip connections can—alternatively or additionally—be used where there are multiple decoder branches. The MLL 3500 can include skip connections 3620 to feed outputs of one or more hidden layers of at least one encoder branch to inputs of corresponding hidden layers of the multiple decoder branches.

Now referring again to FIG. 3, at block 3303, at least one output image is obtained from the MLL 3500 and each one of the at least one output image depicts the tissue sample 3400 including a respective virtual stain.

According to various examples, the MLL 3500 includes multiple decoder branches, such as the decoder branches 3701-3703 shown in FIG. 6 and FIG. 8, and each one of the multiple decoder branches 3701-3703 outputs a respective one of the at least one output images 4001-4003 depicting the tissue sample including a respective virtual stain, e.g. output1, output2 and output3. For example, the three outputs can respectively be virtual H&E (Hematoxylin and Eosin) stained images of the tissue sample, virtually stained images of the tissue sample highlighting antibodies, such as anti-panCK, anti-CK18, anti-CK7, anti-TTF-1, anti-CK20/anti-CDX2, and anti-PSA/anti-PSMA, or other biomarkers. Further examples are primary IHC markers, e.g. HER2 (ERBB2), ER (Estrogen receptor/ESR1), PR (progesterone receptor/PGR); and proliferation markers, e.g. Ki-67 (MKI67). Alternatively, the three outputs can be other virtually stained images depicting the tissue sample including different types of virtual stains. The three different outputs are converted from extracted features of the multiple sets of imaging data 3401-3404 by the three decoder branches 3701-3703, respectively. By using multiple sets of imaging data acquired using multiple imaging modalities as input of the MLL 3500, more relevant information of the tissue sample can be extracted by the at least one encoder branch and correlations across the multiple sets of imaging data are taken into account. Thereby, the at least one decoder branch can generate more precise and robust virtually stained images of the tissue sample. In particular, the MLL 3500 with multiple encoder branches can extract, via a specific encoder branch, specific information of the tissue sample from each individual set of imaging data acquired using a specific imaging modality, and thereby provide imaging modality-dependent information to the at least one decoder branch. Thus, the MLL 3500 can output reliable and accurate virtually stained images. Additionally, the MLL 3500 with multiple decoder branches can output relevant virtually stained images of the tissue sample at once. Further, the MLL 3500 with multiple decoder branches can facilitate reduced computational resources: As intermediate computation results are intrinsically shared within the MLL 3500, the number of logic operations are reduced, e.g., if compared to a scenario in which multiple MLLs are used to obtain output images depicting the tissue sample at different virtual stains.

Above, various scenarios for inference using the MLL 3500 have been described. Prior to enabling inference using the MLL 3500, the MLL 3500 is trained. As a general rule, various options are available for training the MLL 3500. For instance, supervised or semi-supervised learning or even unsupervised learning would be possible. Details with respect to the training are explained in connection with FIG. 9, which shows one example scenario of training. These techniques can be used for training the MLL 3500 described above.

FIG. 9 is a flowchart of a method 3900 according to various examples. FIG. 9 illustrates aspects with respect to training the MLL 3500. For example, the method 3900 according to FIG. 9 may be executed by at least one circuit upon loading program code from a nonvolatile memory. The method of FIG. 9 may be executed by the tissue analyzer 2110. The method facilitates virtual staining of a tissue sample.

The MLL 3500 can be trained using supervised learning, such as the method shown in FIG. 9. The method 3900 is for performing a training of the MLL 3500 for virtual staining. In particular, the method 3900 is used to train the MLL 3500 including at least one encoder branch and multiple decoder branches, e.g., the MLL 3500 of FIG. 6 and FIG. 8. While the scenario FIG. 9 illustrates an architecture of the MLL 3500 including multiple decoder branches, similar techniques may be readily applied to a scenario in which the architecture of the MLL 3500 includes multiple encoder branches.

At block 3901, one or more training images depicting one or more tissue samples are obtained. The training images may be part of one or more sets of training imaging data. For example, as shown in FIG. 10, training images 3911, 3921 are obtained for each of two tissue samples 3910 and 3920, respectively.

The tissue samples 3910 or 3920 can be cancer or cancer-free tissue samples removed from a patient, or tissue samples of other animals or plants. The tissue samples could be in-vivo inspected tissue, e.g., using an endoscope. The tissue samples could be ex-vivo inspected cell cultures.

As illustrated in FIG. 10, it would be possible that multiple instances of the training images 3911 and 3921 are obtained. For example, different sections (i.e., different spatial regions) of the tissue sample or different time instances or even different tissue samples (not illustrated in FIG. 10) may be associated with the multiple instances. Thereby, a larger training database is possible.

While in the scenario FIG. 10 only a single set of training images 3911, 3921 is provided, respectively, as a general rule, it would be possible that multiple sets of training images are obtained, the multiple sets being acquired with multiple imaging modalities. For sake of simplicity, hereinafter, the training process will be described in connection with a single imaging modality providing the training images 3911, 3921, respectively, but the examples can be extended to multiple imaging modalities, e.g., by fusing and using multiple encoder branches as described above.

The method 3900 of FIG. 9 optionally includes—after obtaining the training images 3911, 3921—pre-processing the training images, e.g., using one or a combination of the following image processing techniques: artifacts reduction, such as stitching artifacts, acquisition artifacts, probe contamination, e.g., as air bubbles, dust, etc., registration artifacts, out-of-focus artifacts, etc.; noise filtering; performing a registration 701 between different training images 3911, 3921 (horizontal dotted arrow in FIG. 9); resizing; etc.

At block 3902, multiple reference images 3912-3913, 3922 depicting the one or more tissue samples 3910, 3920 including multiple chemical stains are obtained. The reference images 3912-3913, 3922 serve as a ground truth for training the MLL 3500.

For example, as shown in FIG. 10, for the tissue sample 3910, the reference images 3912-3913 are obtained; and for the tissue sample 3920, the reference image 3922 is obtained. Each reference image 3912-3913, 3922 corresponds to a type of chemical stain, such as H&E, acridine yellow, Congo red and so on. The chemical stains are labeled A, B, and C in FIG. 10. For example, each reference image 3912-3913, 3922 can be obtained by capturing an image of the respective tissue sample 3910, 3920 having been chemically stained using a laboratory staining process that may include, e.g., the tissue specimen being formalin-fixed and paraffin-embedded (FFPE), sectioned into thin slices (typically around 2-10 μm), labelled and stained, mounted on a glass slide and microscopically imaged using, for example, a bright-field microscope.

The reference images 3912-3913, 3922 could be obtained using a fluorescence microscope and appropriate fluorophore. In particular, it is possible to switch on/switch off the respective chemical stain associated with the fluorophore by wavelength-selective excitation. Different fluorophores are excited using different wavelengths and, accordingly, it is possible to selectively excite a given fluorophore. Thereby, it is possible to generate the reference images 3912-3913, 3922 so that they selectively exhibit a certain chemical stain, even if they have been dyed with multiple fluorophores.

Moreover, it is possible to have training images 3911, 3921 which shows a similar structure as the reference images 3912-3913, 3922 by not exciting any fluorophores used to stain the respective tissue sample.

As a general rule, it is not always possible for practical reasons to apply multiple chemical stains to a single tissue sample. For instance, a first reference image may highlight cell cores, another reference image may highlight mitochondrions, due to use of different fluorophores; the different reference images may be acquired from respective columns of a multi-well plate. Thus, multiple reference images may be obtained that depict different tissue samples having different chemical stains. This is why, e.g., the chemical stains of the tissue sample depicted by the reference images 3912-3913, 3922 differ from each other.

Referring again to FIG. 9, at block 3903, the one or more training images 3911, 3921 are processed in the MLL 3500. As the procedure of processing training images is the same as that of processing images of the multiple sets of imaging data, the block 3903 is similar to block 3302 of method 3300. Generally, as shown in FIG. 6 and FIG. 8, the at least one encoder of the MLL 3500 extracts relevant features of the one or more training images and transmits the relevant features to the multiple decoder branches 3701-3703. Each of the multiple decoder branches 3701-3703 converts the relevant features of the one or more training images 3911, 3921 into corresponding training output images 3981-3983, 3991-3993 depicting the respective tissue sample 3910, 3920 including a respective virtual stain, here virtual stains A*, B*, and C* which correspond to chemical stains A, B, C, respectively.

At block 3904, multiple training output images are obtained from the MLL 3500, for each one of the training images. This corresponds to the multi-output scenario. Each one of the multiple training output images is associated with a respective decoder branch and depicts the respective tissue sample including a respective virtual stain. For example, as illustrated in FIG. 10, the training output images 3981-3983 are obtained for the training image 3911; and the training output images 3991-3993 are obtained for the training image 3921.

Generally, as shown in FIG. 6 and FIG. 8, the MLL 3500 includes three decoder branches 3701-3703 of which each decoder branch outputs training output images depicting a tissue sample including a respective virtual stain, here virtual stains A*, B*, and C* which correspond to chemical stains A, B, and C.

After obtaining the one or more training output images 3981-3983, 3991-3993 and the multiple reference images 3912-3913, 3922, the method 3900 optionally includes performing a registration 702-703 between the one or more training output images 3981-3983, 3991-3993 and the multiple reference images 3912-3913, 3922.

As illustrated in FIG. 10, there can be intra-sample registrations 702 and inter-sample registrations 703. The intra-sample registrations 702 between the training output images 3981-3983 and the reference images 3912-3930 that all depict the tissue sample 3910, as well as between the training output images 3991-3993 and the reference image 3922 that all depict the tissue sample 3920. The inter-sample registration 703 can be based on the registration 701 between the training images 3911, 3921.

Such an approach of performing an inter-sample registration 703 between training output images and reference images depicting different tissue samples can, in particular, be helpful where the different tissue samples pertain to adjacent slices of a common tissue probe or pertain to different cell samples of a multi-well plate. Here, it has been observed that the general tissue structure and feature structures are comparable such that the inter-sample registration 703 between such corresponding tissue samples can yield meaningful results. However, other scenarios are conceivable in which a registration between training images and reference images depicting different tissue samples does not yield meaningful results. I.e., inter-sample registration 703 is not always required.

It is not always required to perform all the inter-sample registrations between the training output images 3981-3982 and the reference images 3912-3913, 3922. The method 3900 may optionally be limited to pairwise intra-sample registration 702 between each reference image 3912-3913, 3922 and the multiple training output images 3981-3983, 3991-3993 depicting the same tissue sample 3910, 3920 (vertical dashed arrows).

As mentioned above, there are even scenarios conceivable where the training images and the reference images depict the same structures. This can be the case where the chemical stain is generated from fluorescence that can be selectively activated by using respective excitation light of a certain wavelength. Further, by non-wavelength-selective microscopy, a fluorescence contrast can be suppressed. In such a case, an inter-sample or intra-sample registration is not required, because the same structures are inherently imaged.

Referring to FIG. 9, at block 3905, the training of the MLL is performed by updating parameter values of the MLL based on a comparison between the reference images 3912-3913 and training output images 3981-3983, 3991-3993 that are associated with corresponding chemical stains and virtual stains. The comparison is based on the registrations 702, 703. For instance, a difference in contrast in corresponding spatial regions of the reference images 3912, 3913 and the training output images 3981-3983, 3991-3993 depicting the tissue samples 3910 at corresponding chemical and virtual stains can be determined.

For example, in the scenario FIG. 10, for the tissue sample 3910, the reference image 3912 is compared with the training output image 3981, because the virtual stain A* corresponds to the chemical stain A, e.g., H&E, etc. Likewise, the reference image 3913 is compared with the training output image 3982. For the tissue sample 3920, the reference output image 3922 is compared with the training output image 3993. For each comparison, a respective loss can be calculated. The loss can quantify the difference in contrast between corresponding regions of the training output images and the reference images, respectively.

There is no reference image available for the tissue sample 3910 depicting the tissue sample 3910 including the chemical stain C. Thus, a comparison of the training output image 3983 would only be possible with the reference image 3922 which, however, depicts the tissue sample 3920. Thus, this comparison is only possible if the inter-sample registration 703 between the training output image 3983 and the reference image 3922 is available.

In further detail: With reference to FIG. 6 and FIG. 8, for example, the decoder branches 3701-3703 output training output images 3981-3983, 3991-3993 depicting the tissue samples 3910, 3920 including virtual stains A*, B*, and C*, respectively. The losses of decoder branches 3701-3703 are L1, L2, and L3, respectively, in which L1 is based on a comparison between the training output images 3981, 3991 depicting the tissue samples 3910, 3920 including virtual stain A* and reference images 3912 including chemical stain A, L2 is based on a comparison between the training output images 3982, 3992 depicting the tissue samples 3910, 3920 including the virtual stain B* and reference image 3913 depicting the tissue sample including the chemical stain B, and L3 is based on a comparison between the training output images 3983, 3993 depicting the tissue samples 3910, 3920 including virtual stain C* and reference image 3922 including chemical stain D. The total loss L of the MLL 3500 is a function of L1, L2, and L3, such as L=a1*L1+a2*L2+a3*L3, wherein a1, a2, and a3 are, e.g., manually selected non-negative numbers.

There are various implementations of a loss function possible. For instance, the training of the MLL 3500 may be performed by using other loss functions, e.g., pixel-wise difference (absolute or squared difference) between the reference images 3912-3913 and training output images 3981-3983, 3991-3993 that are associated with corresponding chemical stains and virtual stains; an adversarial loss (i.e., using a generative adversarial network), or smoothness terms (e.g., total variation). Generally, these loss functions can be combined—e.g., in a relatively weighted combination—to obtain a single, final loss function. In some implementations a structured similarity index (https://www.ncbi.nlm.nih.giv/pubmed/28924574) may be used as a loss function.

In such a scenario, because for each tissue sample 3910 all training output images 3981-3983, 3991-3993 are registered to at least one corresponding reference image 391-3913, 3922, the training of the MLL 3500 can jointly update parameter values of at least one encoder branch 3601-3604 and the multiple decoder branches 3701-3703 based on a joint comparison of the multiple reference images and the multiple training output images, such as the loss function L.

As another example, where there is no inter-sample registration 703 available: In such a scenario, the training of the MLL 3500 can include multiple iterations of the method 3900, wherein, for each one of the multiple iterations, the training updates the parameter values of the at least one encoder branch and further selectively updates the parameter values of a respective one of the multiple decoder branches based on a selective comparison of a respective reference image and a respective training output image depicting the tissue same sample including associated chemical and virtual stains. For example, with reference to FIG. 10, the first iteration could only train the decoder branch 3701 providing the training output images depicting the tissue sample including the virtual stain A*. This could be based on a comparison of the training output image 3981 and the reference image 3912 of the tissue sample 3910. Then, the second iteration could only train the decoder branch 3702 providing the training output images depicting the tissue sample including the virtual stain B*. This could be based on a comparison of the training output image 3982 and the reference image 3913 of the tissue sample 3910. Next, the third iteration could only train the decoder branch 3703 providing the training output images depicting the tissue sample including the virtual stain C*. This could be based on a comparison of the training output image 3993 and the reference image 3922. Then, the fourth, fifth, and sixth iteration can proceed with further instances of the respective images 3981, 3912; 3982, 3913; and 3993, 3922. Thus, the training updates the parameter values of the at least one encoder branch, and further selectively updates the parameter values of the decoder branches, such as the decoder branches 3701-3703, based on the total loss L=a1*L1+a2*L2+a3*L3 with {a1; a2; a3} being selected from {1;0;0} or {0;1;0} or {0;0;1}, depending on the currently trained decoder branch 3701-3703.

In some examples, a combination of joint updating of parameter values for multiple decoder branches would be possible, e.g., within each one of the tissue sample 3910 and 3920. In other words, it would be possible to jointly update the parameter values for the decoder branches 3701 and 3702 in a single iteration, because the reference images 3912-3913 depicting the tissue sample 3910 having the virtual stains A* and B* are available with inter-sample registration 702; decoder branch 3703 may be separately updated.

According to various examples of the invention, the multiple iterations are according to a sequence which alternatingly selects reference images and respective training output images depicting the tissue sample including different associated chemical and virtual stains. I.e., the iterations shuffle between different chemical and virtual stains such that different decoder branches 3701-3703 are alternatingly trained. An example implementation would be (A-A*, B-B*, C-C*, B-B*, C-C*, A-A*, C-C*, B-B*, . . . ). A fixed order of stains is not required. For example, this would be different to an approach according to which, firstly, in consecutive iterations all instances of the training output images 3981 are compared with all instances of the reference images 3912, before proceeding to comparing all instances of the training output images 3982 and the reference images 3913. The rationale behind such shuffling through different chemical and virtual stains is to avoid domain-biased training for the at least one encoder branch. For example, where the at least one encoder branch has parameter values that are set based on the comparison is associated with chemical and virtual stain A and A*, only, this can result in parameter values of the encoder branch that are not suited for a domain corresponding to chemical and virtual stain B, B* or C, C*.

Alternatively, according to various examples, the training of the machine-learning logic 3500 includes multiple iterations, wherein, for at least some of the multiple iterations, the training freezes the parameter values of the encoder branches and updates the parameter values of one or more of the multiple decoder branches. Such a scenario may be helpful, e.g., where a pre-trained MLL is extended to include a further decoder branch. Then, it may be helpful to avoid changing of the parameter values of the at least one encoder branch; but rather enforce a fixed setting for the parameter values of the encoder branches, so as to not negatively affect the performance of the pre-trained MLL for the existing one or more decoder branches.

The techniques for training the machine-learning logic 3500 have been explained in connection with a scenario in which the machine-learning logic 3500 includes multiple decoder branches. Similar techniques may be applied to scenarios in which the machine-learning logic 3500 only includes a single decoder branch. Then, it is typically not required to have different samples that illustrate different chemical/virtual stains.

Further, techniques have been described which facilitate training the machine-learning logic 3500 including multiple decoder branches. Similar techniques may be applied to training the machine-learning logic 3500 including multiple encoder branches. Here, as a general observation, typically, it may be possible to obtain reference images as ground truth that depict one and the same tissue sample and that have been acquired using multiple imaging modalities (this is because it is generally possible to measure a tissue sample including a given chemical stain using multiple imaging techniques). However, if this is not the case, then separate encoder branches can be trained separately, as illustrated above in connection with the multiple decoder branches, in particular, by using multiple iterations and defining respective selective loss functions.

Above, some techniques of supervised or semi-supervised learning have been described in which registrations 701-703 between the various images are required. Unsupervised learning would be possible in scenarios in which the chemical stain can be selectively activated using wavelength-selective fluorescence. Then, no registration is required. Further, alternatively or additionally to supervised learning, the MLL 3500 may be trained using a cyclic generative adversarial network (e.g., Zhu, Jun-Yan, et al. “Unpaired image-to-image translation using cycle-consistent adversarial networks.” Proceedings of the IEEE international conference on computer vision. 2017.) architecture including a forward cycle and a backward cycle, each of the forward cycle and the backward cycle including a generator MLL and a discriminator MLL. Both the generator MLLs of the forward cycle and the backward cycle are respectively implemented using the MLL 3500.

Alternatively, the MLL 3500 may be trained using a generative adversarial network (e.g., Isola, Phillip, et al. “Image-to-image translation with conditional adversarial networks.” Proceedings of the IEEE conference on computer vision and pattern recognition. 2017) architecture including a generator MLL and a discriminator MLL. The generator MLL is implemented using the MLL 3500.

The term cycle generative adversarial network as used herein may comprise any generative adversarial network may refer to any generative adversarial network which makes use of some sort of cycle consistency during training. In particular the term cycle generative adversarial network may comprise cycleGAN, DiscoGAN, StarGAN, Dualgan, CoGAN, UNIT.

Examples for such architectures are described in: CycleGAN: see, e.g., Zhu, J. Y., Park, T., Isola, P., & Efros, A. A. (2017). Unpaired image-to-image translation using cycle-consistent adversarial networks. In Proceedings of the IEEE international conference on computer vision (CVPR). DiscoGAN: see, e.g., Kim, T., Cha, M., Kim, H., Lee, J. K., & Kim, J. (2017 August). Learning to discover cross-domain relations with generative adversarial networks. In Proceedings of the 34th International Conference on Machine Learning-Volume 70 (pp. 1857-1865). JMLR. org. StarGAN: See, e.g., Choi, Y., Choi, M., Kim, M., Ha, J. W., Kim, S., & Choo, J. (2018). Stargan: Unified generative adversarial networks for multi-domain image-to-image translation. In Proceedings of the IEEE conference on computer vision and pattern recognition (CVPR). DualGAN: see, e.g., Yi, Z., Zhang, H., Tan, P., & Gong, M. (2017). Dualgan: Unsupervised dual learning for image-to-image translation. In Proceedings of the IEEE international conference on computer vision (ICCV). CoGAN. See, e.g., M.-Y. Liu and O. Tuzel. Coupled generative adversarial networks. Advances in Neural Information Processing Systems (NIPS), 2016. UNIT: see, e.g., Liu, Ming-Yu, Thomas Breuel, and Jan Kautz. “Unsupervised image-to-image translation networks.” In Advances in neural information processing systems (NIPS), 2017.

Summarizing, above, techniques have been described that facilitate implementation of multi-input and/or multi-output scenarios for virtual staining. In particular, scenarios have been described in which a single machine-learning logic can be used. Thereby, a flexibility in the processing of input imaging data is provided, thereby facilitating accurate determining of one or more output images depicting a tissue sample including a virtual stain. Further, by using a single machine-learning logic, it is possible to lower memory consumption. Only a single model needs to be stored after training. The dataset size can be reduced for training. Only a single dataset is required for training, because there is only a single model. Although the single dataset needs to be larger than a dataset for a single output image, it is usually smaller than the combination of datasets of all stains (cf. FIG. 10). Further, computational times can be reduced, and accuracy is improved since correlation across stains are taken into account.

Although the invention has been shown and described with respect to certain preferred embodiments, equivalents and modifications will occur to others skilled in the art upon the reading and understanding of the specification. The present invention includes all such equivalents and modifications and is limited only by the scope of the appended claims.

For illustration, above, various scenarios have been described in which the machine-learning logic configured to output multiple output images depicting the tissue sample including multiple virtual stains is implemented using multiple decoder branches For instance, a conditional neural network may be used. See, e.g., Eslami, Mohammad, et al. “Image-to-Images Translation for Multi-Task Organ Segmentation and Bone Suppression in Chest X-Ray Radiography.” IEEE Transactions on Medical Imaging (2020).

For further illustration, above, various scenarios have been described in which the MLL is implemented by a neural network including at least one encoder branch and at least one decoder branch, wherein the at least one encoder branch provides a spatial contraction and the at least one decoder branch provides a spatial expansion. In some scenarios, it would be possible to use other types of neural networks. For instance, it would be possible to use a neural network that does not implement a spatial contraction and spatial expansion. It has been found that for certain types of virtual stains—e.g., H&E-type virtual stain—a spatial contraction and spatial expansion may not be required, because the visual effect of the stain may be mainly based on morphological features of the biomarker of the tissue. Hence, the respective features may be locally constrained and long-range dependencies are weak. Thus, feature recognition may not rely on long-range dependencies and, thus, spatial contract may not be necessary. Hence, neural networks with a very limited receptive field, e.g., less than 51×51 pixels, could be used. In these scenarios, a neural network could consist of several layers, e.g., convolution, non-linear activation, etc., which keep the number of pixels unchanged. While such models can still be applied to transform input imaging modalities with large numbers of pixels into output imaging modalities of the same pixel number, the prediction of every single pixel in the output will thereby only be based on a spatially limited region on the input imaging modalities.

For still further illustration, various examples have been described for a use case pertaining to histopathology. Similar techniques may be used for other types of tissue samples, e.g., cell microscopy ex-vivo or in-vivo imaging, e.g., for micro-chirurgic interventions. Such techniques may be helpful where, e.g., different columns or rows of a multi-well plate include ex-vivo tissue samples of cell cultures that are died using different fluorophores and thus exhibit different chemical stains. Sometimes, it would be desirable to image a cell culture of a given well of the multi-well plate with multiple stains. Here, stains that are not inherently available chemically, i.e., because the tissue sample in that well has not been stained with the respective fluorophores, can be artificially created as virtual stains using the techniques described herein. This can be based on prior knowledge regarding which chemical stain is available in which well of the multi-well plate. Thus, an image of a tissue sample being stained with one or more fluorophores and thus exhibiting one or more chemical stains can be augmented with one or more virtual stains associated with one or more further fluorophores.

Above, MIMO and SIMO scenarios have been described. Other scenarios are possible, e.g., MISO or single-input single-output SISO scenarios. For instance, for the MISO scenario, similar techniques as described for the MIMO scenario are applicable for the encoder part of the MLL.

1. A method of virtual staining of a tissue sample, the method comprising:
obtaining multiple sets of imaging data depicting a tissue sample, the multiple sets of imaging data having been acquired using multiple imaging modalities,
fusing and processing the multiple sets of imaging data in a machine-learning logic, and
obtaining, from the machine-learning logic, at least one output image, each one of the at least one output image depicting the tissue sample comprising a respective virtual stain.

2. The method of claim 1,
wherein the machine-learning logic comprises at least one encoder branch and at least one decoder branch, the at least one encoder branch providing a spatial contraction of respective representatives of the multiple sets of imaging data, the at least one decoder branch providing a spatial expansion of the respective representatives of the at least one output image,
wherein said fusing is implemented by concatenation of the respective representatives of the multiple sets of imaging data at at least one layer of the at least one encoder branch.

3. The method of claim 2,
wherein the machine-learning logic comprises a single encoder branch,
wherein the at least one layer at which said fusing is implemented comprises an input layer.

4. The method of claim 2,
wherein the machine-learning logic comprises multiple encoder branches, each one of the multiple encoder branches being fed with a respective one of the multiple sets of imaging data,
wherein the at least one layer at which said fusing is implemented comprises at least one hidden layer of the multiple encoder branches.

5. The method of claim 4,
wherein the at least one hidden layer comprises a bottleneck in-between the multiple encoder branches and the at least one decoder branch.

6. The method of claim 1,
wherein the machine-learning logic comprises multiple decoder branches, each one of the multiple decoder branches outputting a respective one of the at least one output image depicting the tissue sample comprising a respective virtual stain.

7. The method of claim 2,
wherein the machine-learning logic comprises multiple encoder branches and skip connections to feed outputs of hidden layers of at least two of the multiple encoder branches to inputs of corresponding hidden layers of the at least one decoder branch, and/or
wherein the machine-learning logic comprises multiple decoder branches and skip connections to feed outputs of hidden layers of the at least one encoder branch to inputs of corresponding hidden layers of the multiple decoder branches.

8. The method of claim 1,
wherein the multiple imaging modalities are selected from the group comprising: hyperspectral microscopy imaging; fluorescence imaging; auto-fluorescence imaging; lightsheet imaging; digital phase contrast; polarization-sensitive acquisition; phase-sensitive imaging; fluorescence lifetime imaging microscopy; Raman spectroscopy; surface-enhanced Raman scattering; stimulated Raman scattering; transmitted-light microscopy; and coherent anti-stokes Raman scattering.

9. A method of performing a training of a machine-learning logic for virtual staining, the machine-learning logic comprising at least one encoder branch and multiple decoder branches, the method comprising:
obtaining one or more training images depicting one or more tissue samples,
obtaining multiple reference images depicting the one or more tissue samples comprising multiple chemical stains,
processing the one or more training images in the machine-learning logic, and
obtaining, from the machine-learning logic and for each one of the one or more training images, multiple training output images, each one of the multiple training output images being associated with a respective decoder branch and depicting the respective tissue sample comprising a respective virtual stain, and
performing the training of the machine-learning logic by updating parameter values of the machine-learning logic based on a comparison between such reference images and training output images that are associated with corresponding chemical stains and virtual stains.

10. The method of claim 9,
wherein the one or more training images depicts a single tissue sample or corresponding tissue samples,
wherein the multiple reference images depict the single tissue sample,
wherein the training jointly updates parameter values of the at least one encoder branch and the multiple decoder branches based on a joint comparison of the multiple reference images and the multiple training output images.

11. The method of claim 10, further comprising:
performing a registration between the multiple training output images and the multiple reference images,
wherein the training is based on the registration.

12. The method of claim 9,
wherein the one or more training images comprises multiple training images depicting multiple tissue samples,
wherein the multiple reference images depict the multiple tissue samples, different ones of the multiple reference images depicting the tissue sample comprising different ones of the multiple chemical stains,
wherein the training comprises multiple iterations, wherein, for each one of the multiple iterations, the training updates the parameter values of the encoder branch and further selectively updates the parameter values of a respective one of the multiple decoder branches based on a selective comparison of a respective reference image and a respective training output image depicting the tissue sample comprising associated chemical and virtual stains.

13. The method of claim 12,
wherein the multiple iterations are according to a sequence which alternatingly selects reference images and respective training output images depicting the tissue sample comprising different associated chemical and virtual stains.

14. The method of claim 12, further comprising:
pairwise registration between each one of the reference images and the multiple training output images depicting the same tissue sample.

15. The method of claim 9,
wherein the training comprises multiple iterations, wherein, for at least some of the multiple iterations, the training freezes the parameter values of the encoder branch and updates the parameter values of one or more of the multiple decoder branches.

16. The method of claim 1,
wherein the machine-learning logic comprises at least one encoder branch and multiple decoder branches, and the machine-learning logic is trained by:
obtaining one or more training images depicting one or more tissue samples,
obtaining multiple reference images depicting the one or more tissue samples comprising multiple chemical stains,
processing the one or more training images in the machine-learning logic, and
obtaining, from the machine-learning logic and for each one of the one or more training images, multiple training output images, each one of the multiple training output images being associated with a respective decoder branch and depicting the respective tissue sample comprising a respective virtual stain, and
performing the training of the machine-learning logic by updating parameter values of the machine-learning logic based on a comparison between such reference images and training output images that are associated with corresponding chemical stains and virtual stains.

17. A device comprising a circuit configured to:
obtain multiple sets of imaging data depicting a tissue sample, the multiple sets of imaging data having been acquired using multiple imaging modalities,
fuse and process the multiple sets of imaging data in a machine-learning logic, and
obtain, from the machine-learning logic, at least one output image, each one of the at least one output image depicting the tissue sample comprising a respective virtual stain.

18. The device of claim 17,
wherein the machine-learning logic comprises at least one encoder branch and at least one decoder branch, the at least one encoder branch providing a spatial contraction of respective representatives of the multiple sets of imaging data, the at least one decoder branch providing a spatial expansion of the respective representatives of the at least one output image,
wherein said fusing is implemented by concatenation of the respective representatives of the multiple sets of imaging data at at least one layer of the at least one encoder branch.

19. The device of claim 18,
wherein the machine-learning logic comprises a single encoder branch,
wherein the at least one layer at which said fusing is implemented comprises an input layer.

20. The device of claim 18,
wherein the machine-learning logic comprises multiple encoder branches, each one of the multiple encoder branches being fed with a respective one of the multiple sets of imaging data,
wherein the at least one layer at which said fusing is implemented comprises at least one hidden layer of the multiple encoder branches.

21. The device of claim 20,
wherein the at least one hidden layer comprises a bottleneck in-between the multiple encoder branches and the at least one decoder branch.

22. The device of claim 18,
wherein the machine-learning logic comprises multiple decoder branches, each one of the multiple decoder branches outputting a respective one of the at least one output image depicting the tissue sample comprising a respective virtual stain.

23. The device of claim 18,
wherein the machine-learning logic comprises multiple encoder branches and skip connections to feed outputs of hidden layers of at least two of the multiple encoder branches to inputs of corresponding hidden layers of the at least one decoder branch, and/or
wherein the machine-learning logic comprises multiple decoder branches and skip connections to feed outputs of hidden layers of the at least one encoder branch to inputs of corresponding hidden layers of the multiple decoder branches.

24. The device of claim 17,
wherein the multiple imaging modalities are selected from the group comprising: hyperspectral microscopy imaging; fluorescence imaging; auto-fluorescence imaging; lightsheet imaging; digital phase contrast; and Raman spectroscopy.

25. A device comprising a circuit configured to perform a training of a machine-learning logic for virtual staining, the machine-learning logic comprising at least one encoder branch and multiple decoder branches, the circuit configured to:
obtain one or more training images depicting one or more tissue samples,
obtain multiple reference images depicting the one or more tissue samples comprising multiple chemical stains,
process the one or more training images in the machine-learning logic, and
obtain, from the machine-learning logic and for each one of the one or more training images, multiple training output images, each one of the multiple training output images being associated with a respective decoder branch and depicting the respective tissue sample comprising a respective virtual stain, and
perform the training of the machine-learning logic by updating parameter values of the machine-learning logic based on a comparison between such reference images and training output images that are associated with corresponding chemical stains and virtual stains.

26. The method of claim 9, further comprising training
the machine-learning logic using a device comprising a circuit configured to perform a training of a machine-learning logic for virtual staining, the machine-learning logic comprising at least one encoder branch and multiple decoder branches, the circuit configured to:
obtain one or more training images depicting one or more tissue samples,
obtain multiple reference images depicting the one or more tissue samples comprising multiple chemical stains,
process the one or more training images in the machine-learning logic, and
obtain, from the machine-learning logic and for each one of the one or more training images, multiple training output images, each one of the multiple training output images being associated with a respective decoder branch and depicting the respective tissue sample comprising a respective virtual stain, and
perform the training of the machine-learning logic by updating parameter values of the machine-learning logic based on a comparison between such reference images and training output images that are associated with corresponding chemical stains and virtual stains.
